(19) United States
(12) Patent Application Publication
(10) Pub. No.: US 2020/0330082 A1
(43) Pub. Date: Oct. 22, 2020

(54) Endoscopic Cannula for Fallopian Tube Access

(71) Applicant: Cruzar Medsystems, Inc., Braintree, MA (US)

(72) Inventors: Albert K. Chin, Palo Alto, CA (US); Michael Glennon, Norwell, MA (US)

(21) Appl. No.: 16/851,954

(22) Filed: Apr. 17, 2020

Related U.S. Application Data
(60) Provisional application No. 62/836,364, filed on Apr. 19, 2019.

(51) Int. Cl. A61B 10/02; A61B 10/04; A61M 25/10

(52) U.S. Cl. CPC A61B 10/0291; A61B 10/04; A61M 2210/1425; A61M 2025/1065; A61M 25/10

(57) ABSTRACT

The present disclosure relates to systems and methods for cell collection within a vessel. The systems and methods can include a cannula having with a pathway extending between a first end and a second end of the cannula and a first lumen situated longitudinally within the pathway of the cannula. The systems and methods can also include an inverted balloon situated within the first lumen, the balloon being coupled at one end to the second end of the cannula and coupled at an opposing end to a rod to permit controlled eversion. In the presence of positive pressure along a defined path diverging from an axis of the pathway the balloon, along with an inverted sleeve situated within the balloon, can evert along the defined path from within the cannula.

### CROSS-REFERENCE TO RELATED APPLICATION(S)

This application claims priority to, and the benefit of, co-pending U.S. Provisional Application No. 62/836,364, filed Apr. 19, 2019, for all subject matter common to both applications. The disclosure of said provisional application is hereby incorporated by reference in its entirety.

### FIELD OF THE INVENTION

This disclosure relates generally to devices and methods for endoscopic access and navigation through the Fallopian tube. More specifically, it relates to devices that may be advanced under endoscopic guidance in an atraumatic fashion to deploy an everting balloon through the Fallopian tube.

### BACKGROUND

Fallopian tube cannulation may be required for diagnostic purposes such as evaluation of infertility, or cell sampling for tubal or ovarian malignancy. If these examinations are required on a routine basis; for example, annual appointments, procedural pain should be minimized and local, regional or general anesthesia may be required to perform Fallopian tube cannulation. Otherwise, the additional cost, time and medical personnel requirements would render the technique prohibitive as a recurring diagnostic procedure. Local anesthesia, such as para-cervical injection of Lidocaine, can be painful to the patient during administration. Similarly, regional anesthesia, such as an epidural block, can cause pain during application. General anesthesia carries a finite risk of patient morbidity and mortality. Intravenous sedation with agents such as Propofol may cause cardiac arrhythmias, organ failure and death. Therefore, there are a number of drawbacks related to conventional diagnostic methods, even when administering anesthesia.

Diagnostic and therapeutic procedures are generally performed on the Fallopian tube via the use of a rigid operative hysteroscope that has a 5 mm or 7 mm outer diameter. The first step in hysteroscopic examination involves insertion of a vaginal speculum to allow visualization of the cervix. Vaginal speculum placement may be moderately painful, however, it generally does not require anesthetic administration. In order to insert the hysteroscope through the cervical os into the uterus, a surgical clamp called a tenaculum is applied to the cervix. The pointed jaws of the tenaculum can cause severe pain upon application; cervical manipulation and hysteroscope insertion cause additional pain. Saline infused under pressure to provide a cavity for hysteroscopic viewing, may also cause cramping pain to the patient. Therefore, it is difficult to perform in-office hysteroscopy on an un-anesthetized patient.

Vaginoscopy is also being performed by gynecologists, using a 5.5 mm rigid hysteroscope to enter and perform therapeutic procedures inside the uterus. Use of a vaginal speculum and tenaculum forceps may be avoided in vaginoscopy, however, intravenous sedation may be required. Saline irrigation is infused via the hysteroscope to distend the vaginal vault and the uterus during the procedure, and the irrigation pressure may cause patient discomfort.

### SUMMARY

There is a need for devices and techniques that may allow access to the Fallopian tube without the need for application of ancillary surgical instruments such as a vaginal speculum, a tenaculum forceps, and a uterine dilator. In particular, devices and techniques are desired that may access the Fallopian tube without incurring injury to the tube, and without causing patient discomfort requiring local, regional or general anesthesia and intravenous sedation for pain control. The present disclosure provides a solution to such a need. Specifically, the present disclosure provides an endoscopic catheter for Fallopian tube cannulation that can be used for an outpatient procedure, such that application of the catheter does not cause patient pain, discomfort, or necessitating anesthetic administration.

In accordance with example embodiments of the present invention, a system for cell collection within a vessel is provided. The system includes a cannula having with a pathway extending between a first end and a second end of the cannula, and a first lumen situated longitudinally within the pathway of the cannula. The system also includes an inverted balloon situated within the first lumen, the balloon being coupled at one end to the second end of the cannula and coupled at an opposing end to a rod to permit controlled eversion in the presence of positive pressure along a defined path diverging from an axis of the pathway. The system further includes an inverted sleeve situated within the balloon and being attached at one end to the second end of the cannula, such that it everts with the balloon along the defined path upon eversion of the balloon from the cannula.

In accordance with aspects of the present invention, a surface of the inverted sleeve includes at least one of a textured surface, an adhesive surface, and an open mesh surface. The balloon can be in fluid communication with the pathway and configured to receive pressurizing fluid from the pathway for eversion out from the second end of the cannula. The sleeve can be configured to evert from the second end of the cannula upon pressurization of the balloon. The system can further include a second lumen within the cannula, the second lumen configured to receive an endoscope.

In accordance with aspects of the present invention, system further includes a transparent hood located at the second end of the cannula, the transparent hood configured to provide viewing from within the second end of the cannula. The cannula can be sufficiently flexible to be advanced through a vessel in a body. The balloon can have an expandable diameter sufficiently large to advance and press the inverted sleeve against inner walls of the vessel for cell collection. The vessel can be a Fallopian tube. The inverted balloon and the inverted sleeve can be configured to move longitudinally within the pathway of the cannula. The cannula can be angled at its distal end in a direction radial away from the cannula to provide the defined path. The second end of the cannula can include an angled rounded tip.

In accordance with example embodiments of the present invention, a method for cell collection within a biological vessel is provided. The method includes placing within the vessel a cannula. The cannula having a pathway extending between a first end and a second end of the cannula and an inferior lumen positioned along the pathway, an inverted balloon situated within the inferior lumen and being coupled to the second end of the cannula, and an inverted sleeve situated within the balloon and being attached to the second end of the cannula, such that upon eversion of the balloon from the cannula, the sleeve is also everted from the cannula. The method also includes everting at least a portion of the inverted sleeve from the second end of the cannula and initiating contact between a surface of the inverted sleeve and a sidewall of the vessel to collect a cell sample onto the surface of the sleeve.

In accordance with aspects of the present invention, the method further includes pressurizing the balloon by inputting fluid into the inferior lumen. The method can further include controlling eversion of the pressurized balloon by controlling an elongated member coupled to a proximal end of the balloon, to allow the balloon to evert longitudinally toward the second end of the cannula. The at least the portion of the sleeve can be everted by the everting balloon. The method of claim 16, wherein the sleeve can be fully everted from the pathway of the cannula. The method can further include inflating a transparent hood located at the second end of the cannula. The method can further include advancing an endoscope in a superior lumen located within the cannula to view the second end of the cannula. The method can further include viewing the vessel via the endoscope through the transparent hood.

### BRIEF DESCRIPTION OF THE FIGURES

These and other characteristics of the present disclosure will be more fully understood by reference to the following detailed description in conjunction with the attached drawings, in which:

FIG. 1 shows an illustrative diagram of conventional insertion of a vaginal speculum;

FIG. 2 shows an illustrative diagram of a conventional rigid hysteroscope advanced towards the uterine cavity;

FIG. 3 shows an illustrative diagram of manipulation of the cervix using a tenaculum;

FIGS. 4A and 4B show illustrative diagrams of a linear everting catheter advanced towards a Fallopian tube containing a proximal pouch, and perforation through the pouch by the everting balloon using conventional systems;

FIGS. 5A, 5B, and 5C show cross-sectional views of the elements contained in the endoscopic cannula for Fallopian tube access, in accordance with the present disclosure;

FIGS. 6A, 6B, 6C, and 6D show cross-sectional views of the elements contained in the endoscopic cannula for Fallopian tube access and the difference in tip visualizations between a straight and an angled tip, in accordance with the present disclosure;

FIGS. 7A, 7B, 7C, and 7D show cross-sectional views of the elements contained in the endoscopic cannula for Fallopian tube access and the increased visual field observed upon inflation of the transparent viewing hood, in accordance with the present disclosure;

FIGS. 8A, 8B, and 8C show an example process implementing the endoscopic cannula of the present invention to access and traverse a Fallopian tube, in accordance with the present disclosure;

FIG. 9 show cross-sectional views of the elements contained in the endoscopic cannula, with a linear eversion balloon catheter inserted through an instrument channel of the endoscopic cannula, for Fallopian tube access, in accordance with the present disclosure;

FIGS. 10A, 10B, and 10C show an example process implementing the endoscopic cannula of the present invention to access and traverse a Fallopian tube, in accordance with the present disclosure; and

FIGS. 11A and 11B show cross-sectional views of the elements contained in the endoscopic cannula, with a linear eversion balloon catheter inserted through an instrument channel of the endoscopic cannula, for Fallopian tube access, in accordance with the present disclosure.

### DETAILED DESCRIPTION

Conventionally, catheter designs can contain a balloon designed for everting from within a cannula or catheter for navigation within a body, such as a Fallopian tube. Specifically, the systems and methods of the present disclosure can be used to navigate within, examine, and treat strictures in the Fallopian tube. Such catheter designs may also be used with or without a hysteroscope. Additionally, in some instances, vaginal speculum and tenaculum application may also be required for insertion of the systems and methods through the cervical os. Catheter designs can also contain a small (0.5 mm diameter) endoscope inside a lumen of an inverted balloon, and upon balloon eversion, the endoscope can be driven forward in the Fallopian tube at twice the rate of the advancing balloon front. The everting balloon can be inflated linearly with substantial pressure, 6-8 atm (88-118 psi), and if left uncontrolled, the pressurized everting balloon may have the potential to perforate the Fallopian tube during passage. For example, a ledge or pouch may be present in the Fallopian tube immediately distal to its os, or opening in the uterus. If the linear everting balloon catheter is directed towards the pouch, the everting balloon can exert sufficient axial force to drive through the back wall of the pouch, rather than deflecting into the true lumen of the Fallopian tube. In addition, the endoscope that resides in the center of the everting balloon can be driven straight forward ahead of the balloon, increasing the potential for the endoscope to perforate the Fallopian tube during deployment. The systems and methods of the present disclosure can be designed to avoid such perforations.

An illustrative embodiment of the present disclosure relates to systems and methods that includes a relatively small diameter (approximately 3 mm-4 mm) cannula that contains a slightly angled distal tip and an endoscope proximal to the slightly angled tip that resides within an inflatable transparent hood. Inflation of the transparent hood can displace vaginal or uterine tissue to increase the field of view for the endoscope without the need for speculum retraction or intrauterine saline distention. The angled tip can be manually inserted through the cervix into the uterus, and subsequently into the Fallopian tube under endoscopic guidance. Once inserted, an eversion balloon can be everted out of the cannula through the Fallopian tube. In some embodiments, the cannula can provide a pathway in which a catheter including an everting balloon can be inserted into the Fallopian tube. Using these design features, the systems and methods of the present disclosure can avoid the need for speculum insertion, uterine tenaculum manipulation, and uterine saline distention, therefore, removing the painful components of Fallopian tube access and cannulation observed in conventional techniques.

In some embodiments, the system can include an everting balloon catheter with a soft distal tip for insertion through a lumen in the cannula. The everting balloon may initially be in a partially everted configuration, such that the partially everted balloon front is substantially flush with the distal end of the soft tip. The pressure in the partially everted balloon can add column strength to the soft tip, to facilitate manual insertion of the first 15 mm length of everting balloon catheter into the Fallopian tube. In some embodiments, the balloon catheter can be implemented without a soft tip and a 10 mm to 15 mm length of inverted balloon is everted distal to the tip of the cannula. Upon pressurization of the inferior lumen, the partially everted balloon itself can form a soft tip that can be manually inserted into the Fallopian tube.

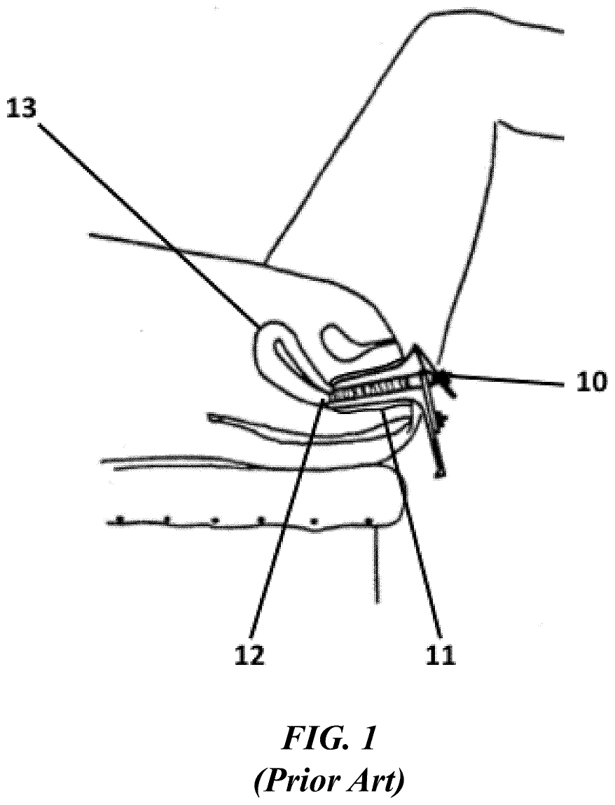

FIGS. 1 through 11B, wherein like parts are designated by like reference numerals throughout, illustrate an example embodiment or embodiments of improved operation for devices and methods for endoscopic access and navigation through the Fallopian tube, according to the present disclosure. Although the present disclosure will be described with reference to the example embodiment or embodiments illustrated in the figures, it should be understood that many alternative forms can embody the present disclosure. One of skill in the art will additionally appreciate different ways to alter the parameters of the embodiment(s) disclosed, such as the size, shape, or type of elements or materials, in a manner still in keeping with the spirit and scope of the present disclosure.

An illustration of an example of a conventional vaginal speculum 10 placed in the vaginal vault 11 of a patient is shown in FIG. 1. The speculum 10 can be designed to retract vaginal tissue to allow visualization of and access to the cervix 12, for entry into the uterus 13. Although it is routinely used for pelvic examinations, patient discomfort is common with its use.

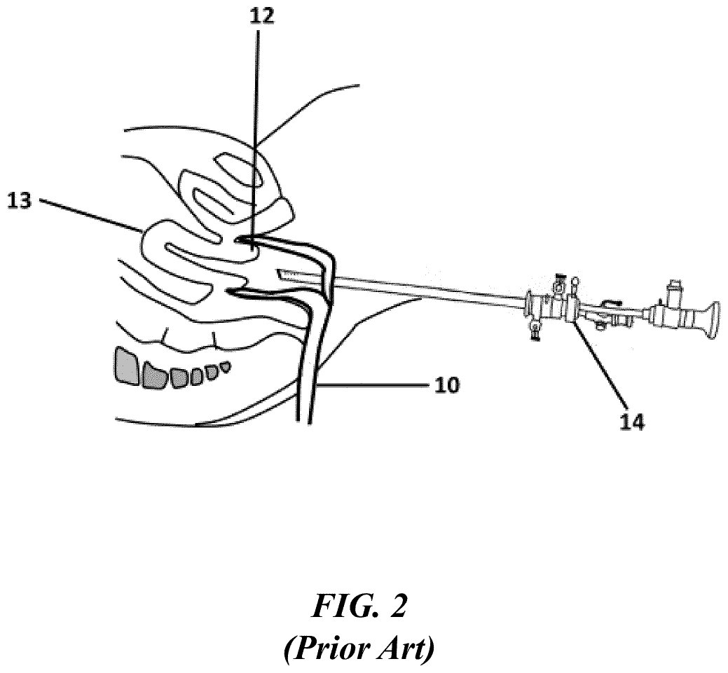

FIG. 2 shows an example of a conventional rigid hysteroscope 14 inserted into the uterus 13. A rigid hysteroscope can generally inserted through the vaginal speculum 10 and inserted through the os of the cervix 12 to examine and treat the uterine cavity. Vaginoscopy involves insertion of a hysteroscope 14 into the uterus 12 without the use of a vaginal speculum 10. This is generally possible only if a small 5 mm diameter hysteroscope is being used, otherwise, substantial pain may be experienced by the patient upon passage of the hysteroscope through the cervical os. Saline irrigation can be introduced through the hysteroscope distends the vaginal vault to allow visualization of the cervix. Upon hysteroscope insertion through the cervical os, saline irrigation distends the uterus to allow visualization of the uterine cavity. The pressurized saline used for uterine distention may cause patient discomfort or pain.

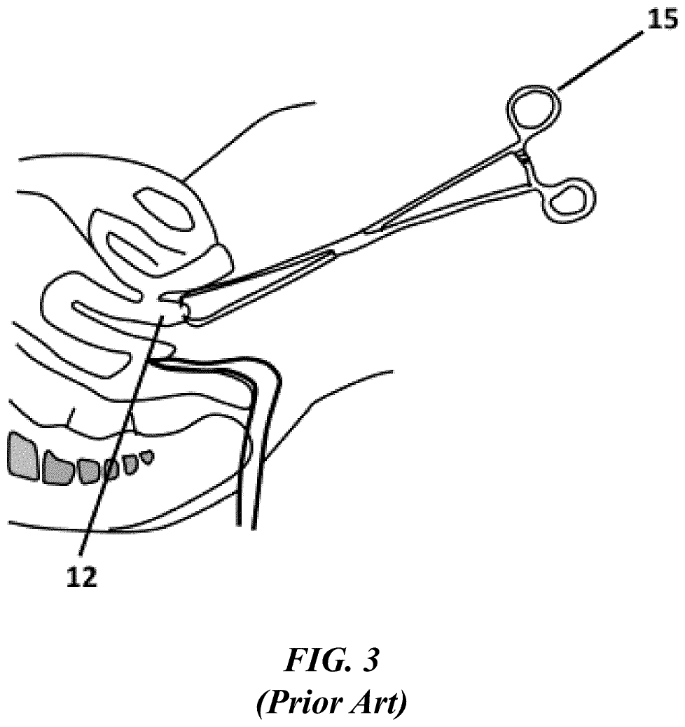

FIG. 3 shows an example of conventional tenaculum forceps 15 typically used to grasp and manipulate the cervix 12 during insertion and use of a hysteroscope. Tenaculum application is well known to cause severe patient pain, and it often requires local anesthesia via a paracervical block, as well as intravenous sedation. Dilation of the cervix may also be necessary to accommodate hysteroscope insertion into the uterus.

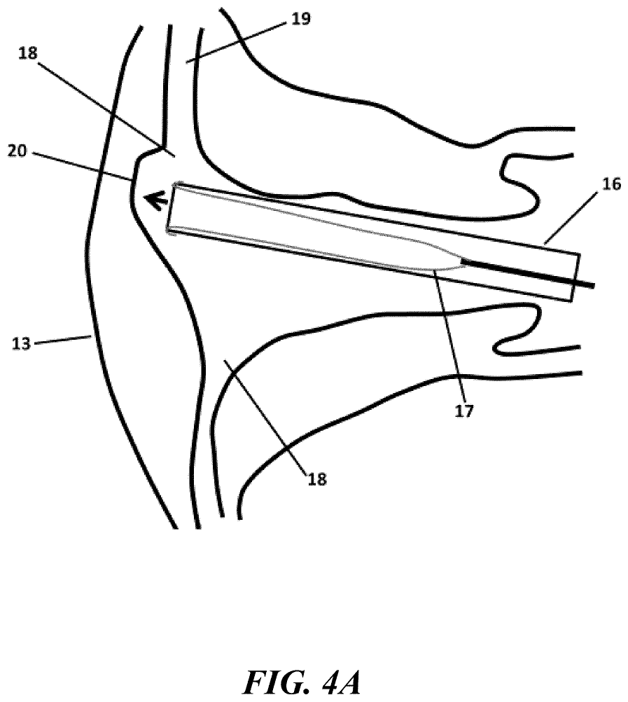

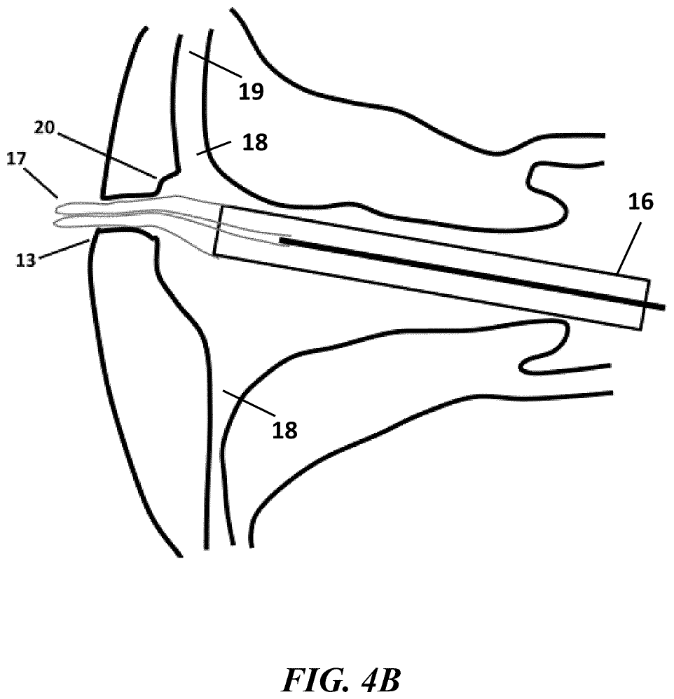

FIG. 4A shows an example of a linear everting balloon catheter 16 that is inserted into the uterus 13 to traverse the Fallopian tube 19. The occurrence of tubal perforation during balloon 17 eversion may occur if an anatomic pouch 20 is present at the opening or os 18 of the Fallopian tube 19. If the linear everting balloon catheter 16 is directed at the pouch 20, and the balloon can be everted under pressure at a level such that it can contain sufficient power to perforate through the pouch 20 and the uterine wall, as shown in FIG. 4B. Specifically, FIG. 4B shows an everting balloon 17 perforating through the pouch 20 and the uterus 13. Tubal and uterine perforation may be serious and lead to complications such as ectopic pregnancy.

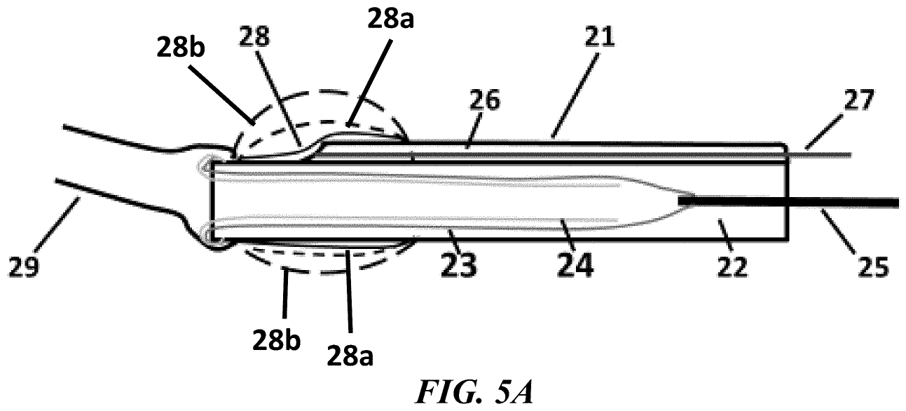

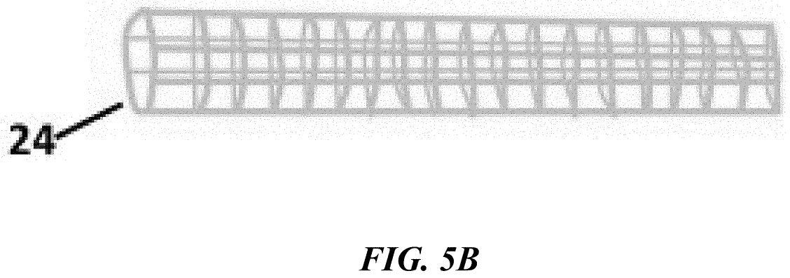

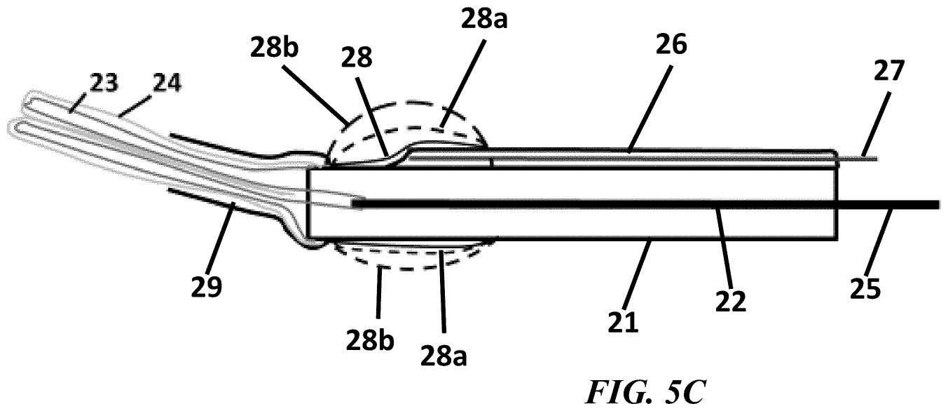

Referring to FIG. 5A, in some embodiments, a system including an endoscopic cannula 21 that can cannulate a vessel, such as a Fallopian tube 19, without patient pain and without the potential for perforation is provided. The cannula 21 can include any combination of sizes, shapes, and materials to facilitate insertion and navigation within a uterus 13 and Fallopian tube 19. For example, the cannula 21 of the present disclosure can be constructed of multi-lumen catheter tubing formed of polyvinyl chloride, polyethylene, Nylon, polyurethane, or similar polymer. The cannula 21 can have continuous a pathway extending between a first end and a second end.

In some embodiments, the cannula 21 can be constructed of multi-lumen polymer tubing, with an inferior lumen 22 and a superior lumen 26 situated longitudinally within the pathway of the cannula 21. The cannula 21 can be sized, shaped, and scaled to be designed for any combination of applications, procedures, or processes. For example, the cannula 21 can be sized and shaped for insertion into a Fallopian tube 19. Similarly, the inferior lumen 22 and a superior lumen 26 can include any combination of sizes and shapes to fit within the cannula 21. In some embodiments, the inferior lumen 22 can be sized and shaped to include an inverted balloon 23 and an inverted sleeve 24 therein while providing sufficient size to enable the balloon 23 to be pressurized for eversion along with the sleeve 24. In some embodiments, the superior lumen 26 can be sized and shaped to include an endoscope 27 be included therein, and optionally enable the endoscope 27 to be able to move within the superior lumen 26.

In some embodiments, the interior portion of the cannula 21 can be separated into at least two sections including the inferior lumen 22 and the superior lumen 26. The inferior lumen 22 can extend approximately 1 cm or greater distally than the other lumens, and can be slightly angled at its distal end. In some embodiments, the inferior lumen 22 can be larger in size than the superior lumen 26 to house an inverted balloon 23 and an inverted sleeve 24. For example, the inferior lumen 22 can be sized and shaped to house and enable traversal of the inverted balloon 23 and the inverted sleeve 24 situated therein. The balloon 23 and sleeve 24 can be designed to follow a path of least resistance out of the cannula 21 and follow a contour of a vessel (e.g., Fallopian tube 19) without causing harm to the vessel walls.

In some embodiments, the inverted balloon 23 can be designed to be everted from within the inferior lumen 22 in response to an application of a positive pressure to the inferior lumen 22. Similarly, the balloon 23 can be designed to be re-inverted into the inferior lumen 22 in response to an application of a negative pressure to the inferior lumen 22. For example, a pressurizing fluid (e.g., gas, liquid, etc.) can be injected and/or withdrawn from the inferior lumen 22 to apply different levels of pressure for controlled eversion and re-inversion of the balloon 23. In some embodiments, because of the relationship between the sleeve 24 and the balloon 23, the sleeve 24 can be everted and/or re-inverted along with the balloon 23 along the defined path upon eversion and/or re-inversion of the balloon 23 from the cannula 21. Although the inverted sleeve 24 is described to invert with a balloon 23 and or a rod 25, the inverted sleeve 24 can be designed to evert and/or re-invert from the cannula 21 without the use of the balloon 23 and or the rod 25.

In some embodiments, at least one end of each of the inverted balloon 23 and the inverted sleeve 24 can be coupled to the distal end of the cannula 21. The inverted balloon 23 and the inverted sleeve 24 can be coupled to the cannula 21 using any combination of methods. For example, the inverted balloon 23 and the inverted sleeve 24 can be welded, adhered, mechanically coupled to the exterior or interior surface of the cannula 21 body. In some embodiments, the sleeve 24, in its inverted state, can be situated within the inverted balloon 23 with an end wrapped around the end of the inverted balloon 23 such that an end of the inverted balloon 23 is positioned between the cannula 21 and the coupled end of the inverted sleeve 24. The inverted balloon 23 and the inverted sleeve 24 can be coupled to the cannula 21 individually, coupled indirectly to the cannula 21, coupled to one another, or a combination thereof. In some embodiments, one end of the inverted sleeve 24 can be uncoupled and free-standing within the inverted balloon 23.

The inverted balloon 23 and the inverted sleeve 24 can be constructed from any combination of material that allows flexible inversion and eversion from within the inferior lumen 22. For example, the inverted balloon 23 can be constructed of a thin-walled (approximately 0.25-0.35 mils) inelastic polymer such as PET (polyethylene terephthalate) or Nylon 12, and inverted sleeve 24 can be constructed of an equally thin porous fabric or fiber meshwork, composed of polyester, silk, or similar material. As would be appreciated by one skilled in the art, the balloon 23 and sleeve 24 can be constructed from any combination of materials known in the art without departing from the scope of the present disclosure.

In some embodiments, the proximal end of balloon 23 can be attached to a rod 25, tube or elongated member positioned within the inferior lumen 22. The rod 25 can be used to control the eversion of balloon 23 upon pressurization of inferior lumen 22. For example, the rod or a tube 25 can be advanced distally in the inferior lumen 22 to regulate the advancement of the balloon 23 during eversion under inflation pressure. In other words, the rod or tube 25 can advanced forward within the inferior lumen 22 for controlled eversion of balloon 23 and delivery of inner sleeve 24. The rod 25 can be attached to the balloon 23 using any method known in the art, for example, welding, adhesive, mechanically coupling, etc. Similarly, the rod 25 can be constructed from any combination of materials to provide sufficient longitudinal rigidity to assist in the eversion of the balloon 23.

Continuing with FIG. 5A, in some embodiments, the superior lumen 26 can be positioned within the cannula 21 and/or coupled to the cannula 21 parallel to the inferior lumen 22. In some embodiments, the superior lumen 26 can be sized and shaped to house an endoscope 27, such as a small diameter 0° endoscope. The endoscope 27 can include any combination of instruments which can be introduced into the body to give a view of its internal part. For example, the endoscope 27 can be a CMOS (complementary metal-oxide-semiconductor) chip endoscope 27 approximately 0.5 mm-1.0 mm in diameter, with incorporated LED (light emitting diode) illumination. The end of superior lumen 26 can be sized and shaped to accommodate views for the endoscope 27 through an end of the cannula 21. For example, the distal end of the superior lumen 26 can be tapered to provide a clear view of the endoscope 27 therein. The tapered end of the superior limen 26 can be provided to enhance visualization (e.g., via the endoscope 27) to see past the distal end of the cannula 21, for example, to see an os 18. Additionally, the sloped end of the superior limen 26 can assist in provided improved views when the cannula 21 is rotated.

In some embodiments, an angled tip 29 can extend from or otherwise be attached to the straight portion of the cannula 21 tip. The angled tip 29 can be any combination of geometric shapes that enable ease of advancement into a vessel (e.g., Fallopian tube 19). For example, the angle tip 29 can be angled, tapered, cylindrical, conical, etc. In some embodiments, the angled tip 29 can contain a rounded distal tip and provide an open channel extending from the end of the inferior lumen 22 of cannula 21 to allow the balloon 23 and sleeve 24 to extend therethrough.

In some embodiments, the angled tip 29 can extend at an angle away from the central axis of the cannula 21. In particular, the angle can be sufficiently angled in a radially away from the axis of the cannula 21 to be used to visualize and cannulate the os 18 to help get through the cervix. For example, the angled tip 29 can extend radially from the end of the cannula 21 and can be angled upward at a sufficient angle such that a 0° endoscope may visualize an entire length of the angled tip 29 in perspective, allowing the physician to precisely insert the angled tip 29 into an opening, such as the Fallopian tubal os 18. The angled tip 29 can be angled in any combination of directions, for example it can be angled radially, in the, inferior, superior, etc. direction at an angle of approximately 5°-10° or more. In contrast, in a straight, non-angled tip, visual appreciation of the tip 29 extending distal to the endoscope 27 may be lost, compromising control of the tip insertion process.

In some embodiments, the angled tip 29 can be designed to be substantially straight during insertion of the cannula 21 into a body then can be adjusted to undertake an angled shape when at a desired location. In particular, the angled tip 29 can go in with straight cannula 21 then can cause the tip 29 and upon positioning within a target location it can assume angled shape extending radially away from the central axis of the cannula 21 body. For example, the tip 29 can have shape memory, can have an outer sleeve to covering and confirming the tip 29 to a straight shape, or a combination thereof that causes the tip 29 to return to angled shape when removed. In another example, the angled tip 29 can have varied flexibility throughout the tip 29 such that it can flex to an angled shape in a particular location.

The angled tip 29 can be coupled to a distal end of the cannula 21 proximal to the distal end of the inferior lumen 22 with the attached everting balloon 23 using any combination of methods. For example, the angled tip 29 can be an extension of the cannula 21 or it can be welded, adhered, mechanically coupled to the exterior surface of the cannula 21 body. In some embodiments, an end of the angled tip 29 coupled to the cannula 21 can extend over the ends of the inverted balloon 23 and the inverted sleeve 24 coupled to the cannula 21 such that the ends of the inverted balloon 23 and the inverted sleeve 24 are positioned between the angled tip 29 and the cannula 21. The angled tip 29, balloon 23, sleeve 24 can be individually, coupled indirectly to the cannula 21, coupled to one another, or a combination thereof.

Similarly, the angled tip 29 can be designed with any combination of sizes. For example, the angled tip 29 can be approximately 10 mm-15 mm in length, and approximately 0.8 mm in outer diameter. In some embodiments, an everting balloon 23 can be contained inside the lumen of the angled tip 29. The angled tip 29 can be constructed from any combination of materials that is sufficiently flexible for navigation within a body while being sufficiently rigid to maintain its angled shape when the balloon 23 and sleeve 24 are everted therethrough, as discussed in greater detail herein. For example, the angled tip 29 can be constructed of a polymer tubing of a lower durometer than the rest of the cannula 21, so that it has less potential of injuring the Fallopian tube os 18 during cannula 21 tip insertion.

In some embodiments, the angled tip 29 can be designed for manual insertion into the os 18 of the Fallopian tube 19 before the balloon 23 is everted the length of the Fallopian tube 19. Using this design avoids everting balloon 23 perforation of the Fallopian tube 19 and the uterotubal junction if a pouch 20 is present immediately distal to the os 18. Specifically, manual insertion of the full length of the angled tip 29 ensures that the distal portion of the tip 29 has advanced beyond the pouch 20 at the ostial region, and balloon 23 eversion may proceed without the potential of perforation. For example, the angled tip 29 can be guided into the os 18 using visual confirmation (or other method) and manipulation of the cannula 21 from outside the body (e.g., endoscopic, fluoroscopic, etc.) can be used to insert the angled tip 29 into the os 18 of the Fallopian tube 19.

In some embodiments, an elastomer hood 28 can be coupled to the distal end of the cannula 21 slightly proximal to the tip of the endoscope 27 within the superior lumen 26. In some embodiments, the proximal end of the hood 28 can attached to the cannula 21 body and the distal end of the hood 28 can be attached to the proximal portion of an angled tip 29, which can be coupled to a distal end of the cannula 21. The hood 28 can be coupled to the cannula 21 and/or the angled tip 29 using any combination of methods. For example, the inverted balloon 23 and the inverted sleeve 24 can be welded, adhered, mechanically coupled to the exterior surface of the cannula 21 body. The elastomer hood 28 can be provided in a deflated state during insertion and an inflated state during endoscopic viewing.

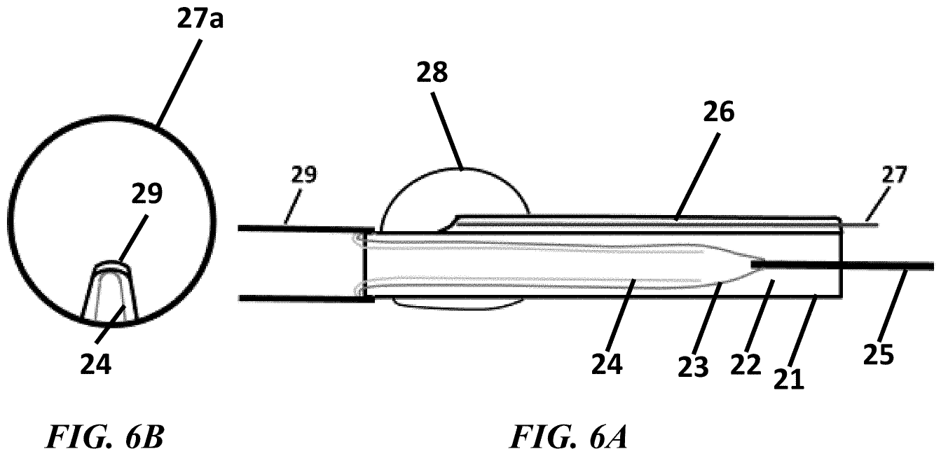

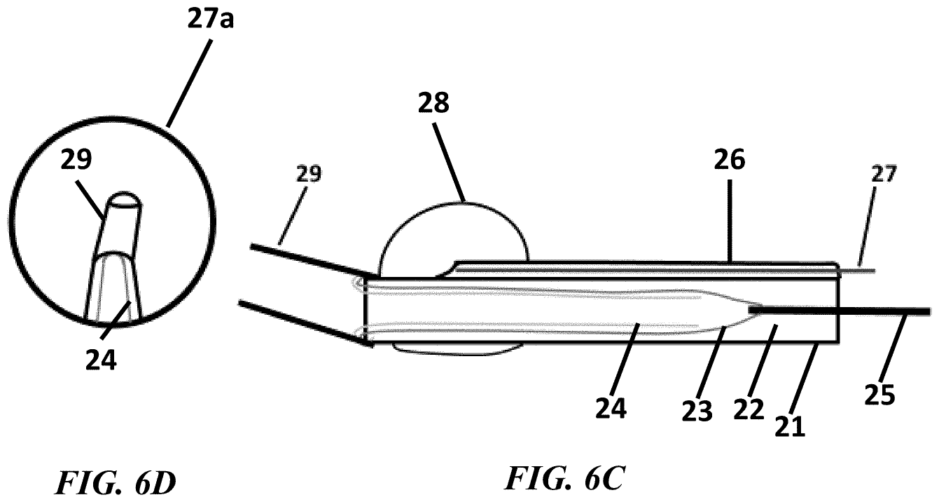

In some embodiments, the hood 28 can be inflated to a size and shape to provide to clear an area around the distal end of the superior lumen 26 to provide a clear line of sight for the endoscope 27 within the superior lumen 26. For example, when inflated, the hood 28 can expand into a substantially round or spherical shape. In some embodiments, to improve visibility for the endoscope 27, the hood 28 can be tapered and/or transparent. The hood 28 can be constructed from any combination of materials, textures, finishes, etc. to provide a substantially transparent window for viewing by the endoscope. For example, a transparent hood 28 can be constructed of polyurethane or silicone rubber, and it may be flexible or elastic. The hood 28 can also be designed to provide a transparent viewing window when in an inflated state. In some embodiments, the hood 28 can be designed such that an upper portion the of the hood 28 can expand to a greater size than the lower portion, as shown in FIG. 6B. Although any combination of hood 28 shapes can be used without departing from the scope of the present invention. For example, the hood can be only located on a top portion of the cannula 21.

In some embodiments, the hood 28 may be inflated via a separate lumen (not depicted) in the cannula 21 or it can be inflated via the inferior lumen 22 with the balloon 23. The hood 28 can be in a deflated state, as shown by hood 28 in FIG. 5A, a partially inflated state 28a, or fully inflated state 28b. When in the fully inflated state 28b, the hood 28 can provide a sufficiently large viewing surface for the endoscope 27 as the cannula 21 is guided around the vaginal vault to cannulate the cervix. For example, when in a fully inflated state 28b, the diameter of the hood 28 may be approximately 20 mm. In some instances, once inside the uterus, minimal hood 28 inflation can be used to distend the uterine cavity to allow visualization of the Fallopian tube os 18, and cannulation of the os 18 with the angled cannula tip 29, as the uterine cavity is approximately 5 cc in volume.

Referring to FIG. 5B, in some embodiments, the inverted sleeve 24 can be constructed from a mesh material. The mesh may be formed of fibers that provide increased friction against the inner surface of the Fallopian tube 19 when sleeve 24 is deployed by the everting balloon 23. This can be particularly useful when the sleeve 24 is used to collect cells from the Fallopian tube 19 for diagnostic purposes (e.g., to sample for ovarian cancer cells that may be present in the Fallopian tube 19). The open lattice structure of a mesh can also enhance cell collection that surpasses present techniques for Fallopian tube 19 cell collection using the smooth, slippery surface of an everted PET or Nylon balloon. For example, the balloon 23 can be everted causing sleeve 24 to evert and initiate contact between a surface of the inverted sleeve and a sidewall of the Fallopian tube 19 vessel to collect a cell sample onto the surface of the sleeve 24. As would be appreciated by one skilled in the art, any combination of surfaces configured for cell sampling can be utilized for the sleeve 24 without departing from the scope of the present disclosure.

In some embodiments, if the endoscopic cannula 21, can be used for collection of cell specimens from the Fallopian tube 19, a telescopic tube (not depicted) may lie inside the inverted balloon 23. When the balloon 23 everts through the Fallopian tube 19, it can deliver the telescopic tube, which then lies outside the everting balloon 23. The everting balloon 23 can be composed of polymeric material, containing a smooth, slippery surface that is unfavorable for cell collection. In some embodiments, the telescopic tube can be used in addition to or in place of the sleeve 24 and/or the balloon 23. The telescopic tube may be formed of a thin fabric material such as cotton, polyester or silk, that imparts a better surface for cell adherence. The telescopic tube may also be a mesh structure formed of thread strands, with an open lattice structure conducive to cell collection and cell retention.

Referring to FIG. 5C, a cross sectional view of the system 100 with a partially everted balloon 23, with the sleeve 24 residing on the outside surface of balloon 23 is depicted. As shown in FIG. 5C, in the everted state, the proximal end of the sleeve 24 can be positioned outside the end of the cannula 21 between two portions of the everted balloon 23. In this orientation, the sleeve 23 can encompass the balloon 23 such that the sleeve 24 separates the everted balloon 23 from the exterior environment (e.g., within a body). With the balloon 23 and sleeve 24 everted, the balloon 23 can have a diameter sufficiently large to advance and press the sleeve 24 in position to contact the inner surface of a vessel (e.g., Fallopian tube 19) for cell collection, dilation, expansion, or any other purpose.

Cannulation of the Fallopian tube 19 requires insertion of the cannula tip 29 into the os 18 of the Fallopian tube 19. When the cannula tip 29 is in a coaxial orientation with endoscope 27, for example as provided in FIGS. 4A and 6A, it is difficult to visualize tip 29 in the endoscopic field of view 27a depicted in FIG. 6B. The length of tip 29 appears foreshortened, and visual control of cannula 21 insertion is compromised.

Referring to FIG. 6C, in contrast, using the system discussed with respect to FIGS. 5A-5C, the angled tip 29 can be used. The slight angulation (approximately 5°-10°) of the tip 29 in the direction of the endoscope 27 can be used to provide a visual perspective of the length of the tip 29, allowing guidance of angled tip 29 into the os 18 of the Fallopian tube 19. FIG. 6D demonstrates an enhanced endoscopic field of view 27a of angled tip 29 when implementing the angled tip 29.

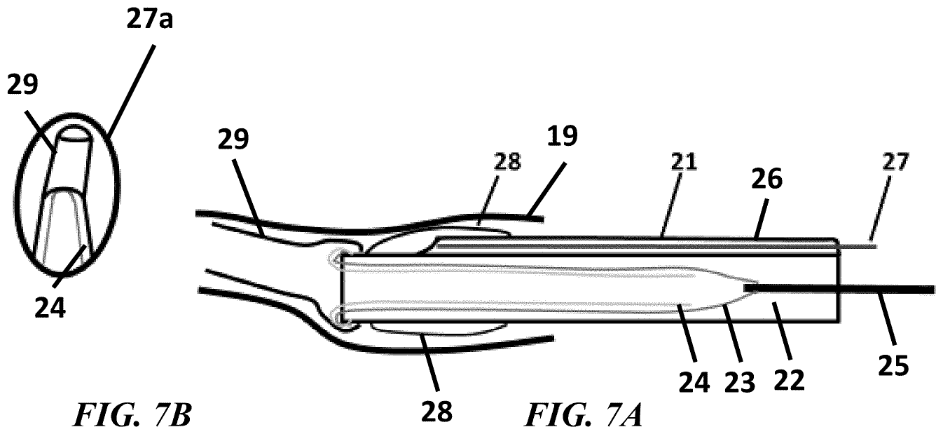

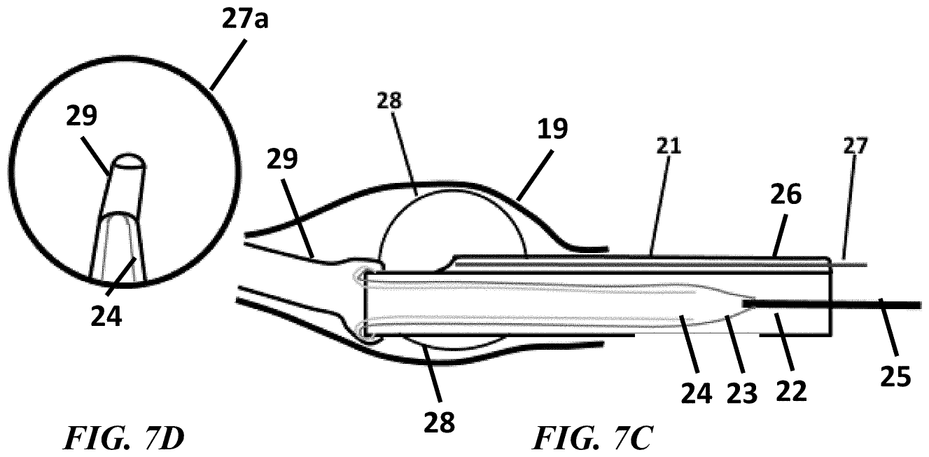

Referring to FIGS. 7A-7D, in some embodiments, enhanced endoscopic visualization can also be provided by the inflatable transparent hood 28 that covers the distal end of endoscope 27. In FIG. 7A, an uninflated hood 28, can result in a narrow endoscopic field of view 27a, as shown in FIG. 7B. Upon inflation of the transparent hood 28, as shown in FIG. 7C, an expanded endoscopic field of view 27a, as shown in FIG. 7D, can be provided. The larger diameter viewing surface shown in endoscopic field of view 27a of FIG. 7D is provided by the inflated hood 28. As shown in FIG. 7C, the hood 28 expands to create a gap between an anatomic tissue draped over the hood 28 is retracted with a resultant wide field of view. The gap created by the hood 28 provides the increased field of view for the endoscope 27 positioned within the superior lumen 26 and directed toward an interior of the hood 28.

In some embodiments, transparent hood 28 can be inflated when cannula 21 is inserted into the vagina, allowing visualization and cannulation of the cervix. For example, any combination of fluid (e.g., gas, liquid, etc.) can in injected through a lumen into the hood 28 to inflate the hood 28. Similarly, fluid can be removed from the hood 28 to deflate the hood. A smaller volume of inflation can be used for visualization inside the uterus, as the uterine cavity is much smaller than the vaginal vault. The inflatable transparent hood 28 can be used to provide limited tissue retraction, as opposed to the generalized saline injection used in conventional hysteroscopy. Generalized saline injection pressurizes the entire organ, can result in the cramping pain experienced by the patient during conventional hysteroscopy. This pain, which may be severe, can be decreased with the use of localized tissue retraction provided by inflatable hood 28.

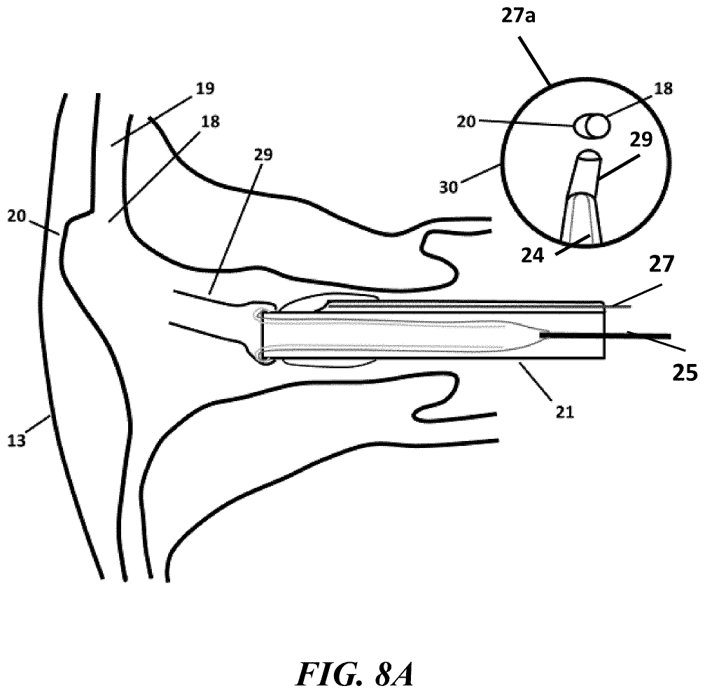

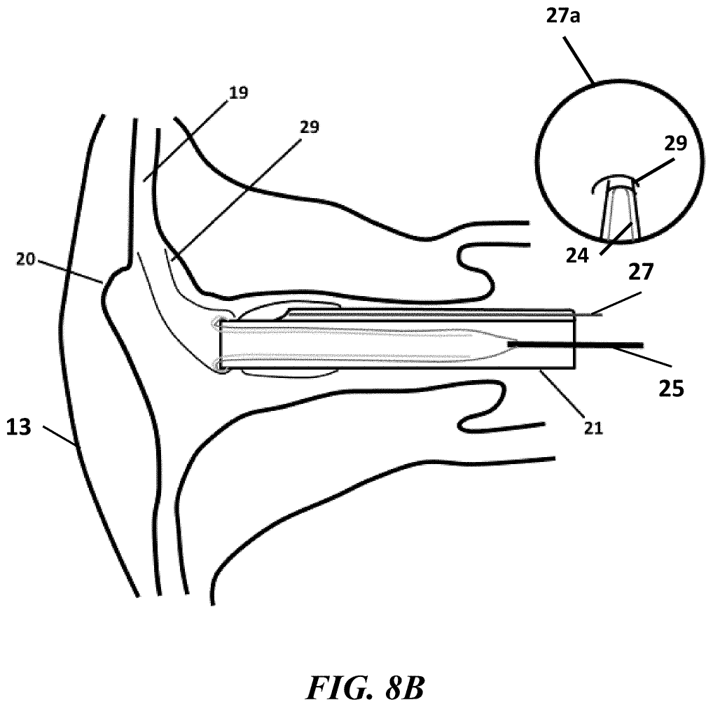

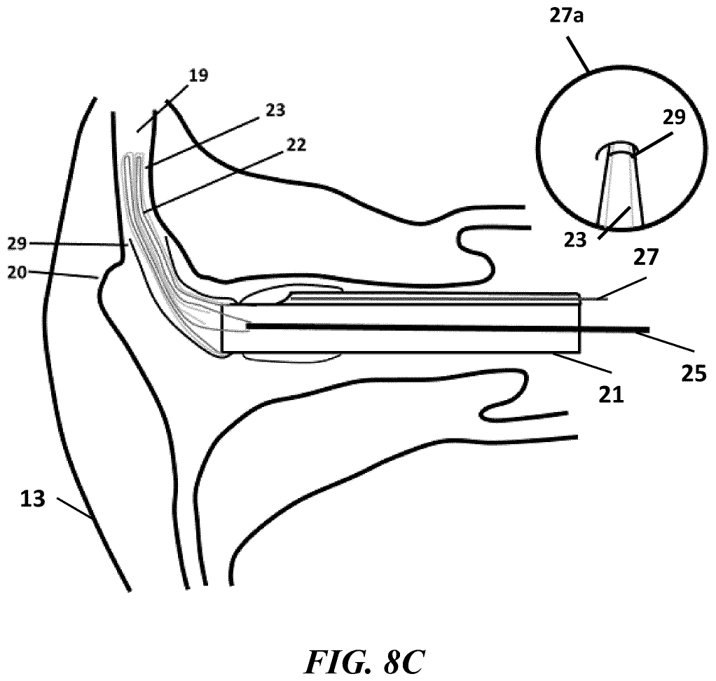

Referring to FIGS. 8A-8C, an exemplary operation of Fallopian tube cannulation, using the system discussed with respect to FIGS. 5A-5C, 6C-6D, and 7C-7D, is provided. Initially, as shown in FIG. 8A, endoscopic cannula 21 can advanced through the uterus 13 towards the os 18 of the Fallopian tube 19. During the initial advancement, the balloon 23 and the sleeve 24 can be inverted within the inferior lumen 22 and the hood 28 can be in a deflated state outside of the cannula 21. In some instances, the hood 28 can be partially inflated to provide a viewing window for the endoscope 27. The endoscopic field of view 27a of FIG. 8A shows the outline of the tubal os 18 and a pouch 20 adjacent the os 18. The hood 28 can be inflated, partially inflated, or deflated at any point in time during any combination of functions.

In some embodiments, the hood 28 can be used in combination with the endoscope 27 for guiding the cannula 21 into position and angle the tip 29 into a particular location (e.g., os 18). For example, under endoscopic guidance, using the endoscopic field of view 27a provided by the endoscope 27, the angled tip 29 can be guided and inserted into the os 18 its full length of approximately 15 mm, as shown in FIG. 8B. Using the endoscopic view 27a, visualization of full-length tip insertion can be used to verify that tip 29 has proceeded into the Fallopian tube 19, instead of being caught in pouch 20. Although the present disclosure provides examples of positioning and using the systems and methods with a endoscopic visualization, any combination of confirmation methods can be used. For example, the systems and methods of the present invention can use direct visualization and/or using fluoroscopy or any other combination of methods

In some embodiments, with the tip 29 in place within the Fallopian tube 19, the balloon 23 and sleeve 24 can be everted from within the inferior lumen 22. For example, a fluid can be injected into the inferior lumen 22 to advance the balloon 23 and sleeve 24 in a distal direction out of the inferior lumen 22 and evert out of the distal end of the inferior lumen 22. Additionally, the rod 25 can be used to assist in both the advancement and controlled a rate in which the balloon 23 and sleeve 24 are everted out of the inferior lumen 22. For example, the rod 25, to which the balloon 23 is attached, can be advanced in a distal direction through the inferior lumen 22. FIG. 8C shows balloon 23 and sleeve 24 everting out of the cannula 21 and the tip 29 into Fallopian tube 19, without the concern that the eversion may cause perforation through pouch 20. The eversion can occur without perforation through pouch 20 because of the combination of the angle of the tip 29 and the controlled eversion by the rod 25. The preferred technique involves manual advancement of angled tip 29 into Fallopian tube 19, followed by eversion of balloon 23 and sleeve 24 the length of tube 19.

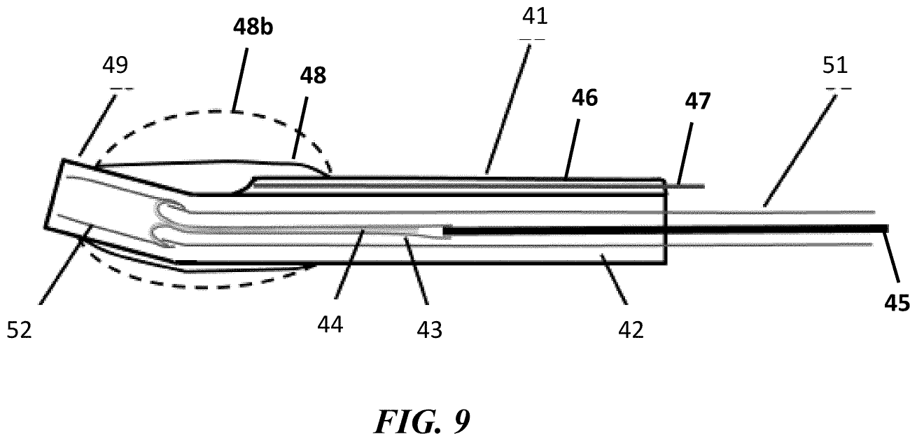

Referring to FIG. 9, in some embodiments, the system can include an everting balloon catheter 51. The everting balloon catheter 51 may be a separate unit that is inserted through the inferior lumen 42 that has an angled distal end 49, as shown in FIG. 9. For example, the angled tip 49 can extend radially from the end of the cannula 41 and can be angled upward at a sufficient angle such that a 0° endoscope may visualize an entire length of the angled tip 49 in perspective, allowing the physician to precisely insert the angled tip 49 into an opening, such as the Fallopian tubal os 18. In some embodiments, the angled tip 49 can be an extension of the inferior lumen 42 itself. The cannula 41 can be designed to receive the everting balloon 43, sleeve 44, and rod 45 as part of and/or through a separate catheter 51 component. For example, the everting balloon 43, sleeve 44, and rod 45 can be part of the separate catheter 51 that can be advanced through the inferior lumen 42 of cannula 41. Other than the separate catheter 51 component, the components provided in FIG. 9 can be similar in design and function as discussed with respect to the cannula 21. For example, the cannula 41 can include an inferior lumen 42, a superior lumen 46 housing an endoscope 47, and a transparent hood 48 similar to those same components 22, 26, 27, 48, etc. discussed with respect to cannula 21. In some embodiments, the distal end of the hood 48 can be coupled to the angled distal end 49 of the inferior lumen 42. Similarly, with the exception to the relationship with the catheter 51, the balloon 43, sleeve 44, and rod 45 can be similar in design and function as the balloon 23, sleeve 24, and rod 55 discussed with respect to FIGS. 5A-8C.

The catheter 51 can include any combination of catheters that are sized and shaped to be inserted within the inferior lumen 42 and receive the balloon 43, sleeve 44, and rod 45. In some embodiments, the catheter 51 can include an angled tip 52 extending radially from the axis of the catheter 51. For example, the catheter 51 can be flexible tube catheter with a 10 mm-15 mm long distal tip 52 beyond the inverted balloon 43 portion of the catheter 51. The angled tip 52 can shape a similar angle as that of angled tip 49 of the cannula 41. The catheter 51 and/or the angled tip 52 can be composed of a lower durometer material, such as polyurethane or silicone rubber. In some embodiments, the distal tip 52 of the catheter 51 can be sufficiently flexible for enhanced atraumatic insertion into the os 18 of the Fallopian tube 19. The distal tip 52 may be a separate component that is bonded onto a distal tip of the catheter 51. In some embodiments, an end of the angled tip 52 coupled to the catheter 51 and can extend over the ends of the inverted balloon 43 and the inverted sleeve 44 coupled to the catheter 51, such that the ends of the inverted balloon 43 and the inverted sleeve 44 are positioned between the angled tip 52 and the catheter 51.

Using the combination of the cannula 41 and the catheter 51, in some embodiments, the angled tip 49 of the cannula 41 may not be inserted into the Fallopian tube 19. Instead, the angled tip 49 may remain at the ostia 18 of the Fallopian tube 19 as a length of the angled tip 52 (e.g., 10 mm to 15 mm) of the everting balloon catheter 51 is inserted into the Fallopian tube 19 past any potential proximal pouch 20 in the Fallopian tube 19. Thereafter, the balloon 43 and the sleeve 44 can be everted out of the angled tip 52 of the catheter 51. For example, the soft tip 52 can be manually advanced its full length into the Fallopian tube 19, followed by full length balloon 43 eversion through the Fallopian tube 19.

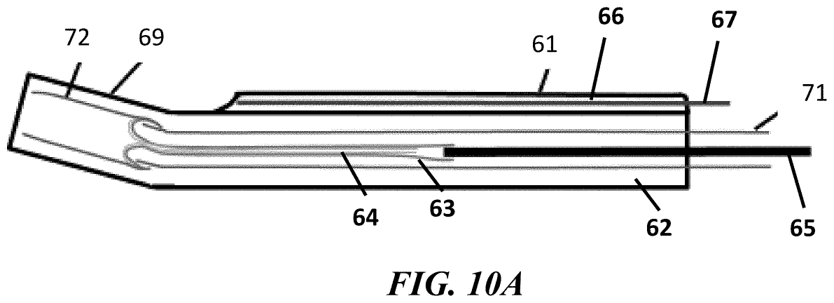

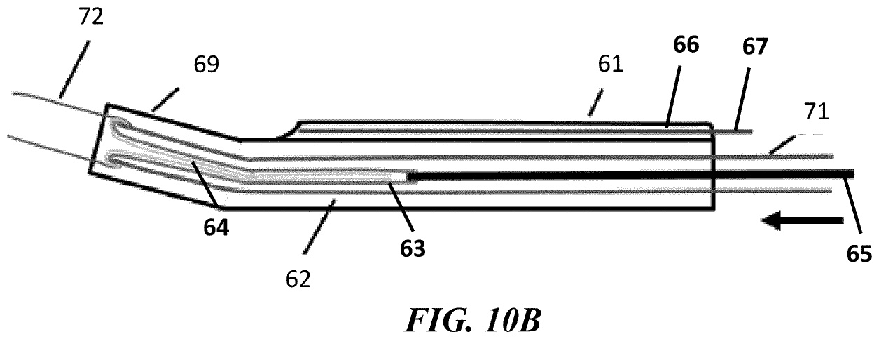

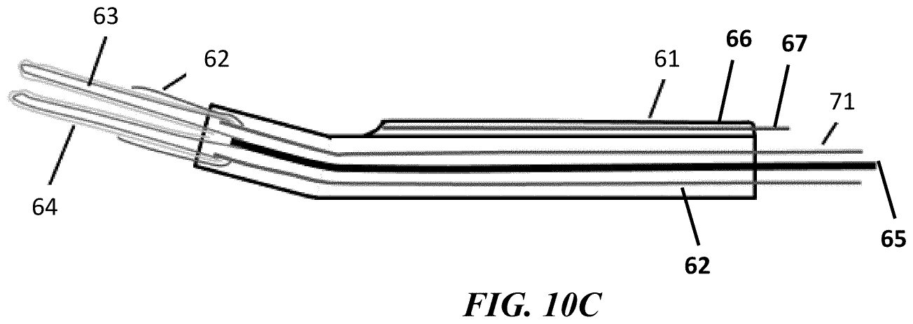

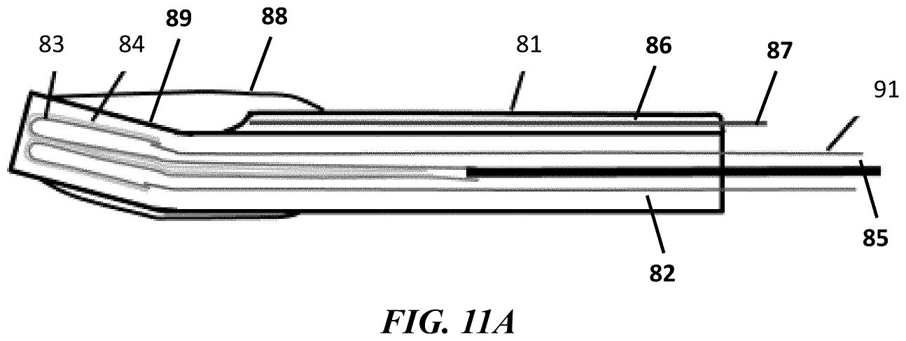

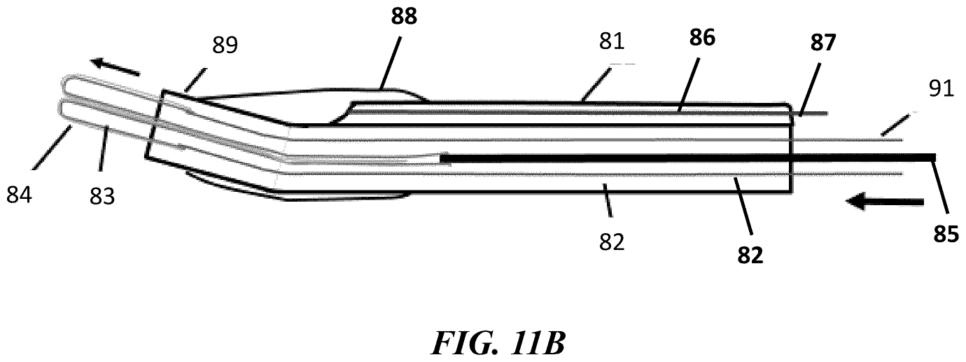

FIG. 10A through 10C show the sequence of steps in the use of a cannula 61 using an everting catheter 71. The cannula 61, separate catheter 71, and components thereof can be similar in design as discussed with respect to the components of cannulas 21, 41 and catheter 51. For example, the cannula 61 can include an inferior lumen 62, a superior lumen 66 housing an endoscope 67, and an angled tip 69, similar to those components discussed with respect to cannulas 21, 41. The systems and methods, as depicted in FIGS. 10A-10C, the cannula 61 can be implemented with or without the use of a hood 28. Similarly, the sequence of steps discussed with respect to FIGS. 10A-10C can be applied to the systems discussed with respect to FIGS. 9 and 11A-11B.

Referring to FIG. 10A, a cannula 61 in its initial position, with soft tip 72 of catheter 71 positioned inside the angled distal tip 69 of cannula 61 is depicted. For example, the catheter 71, with the balloon 63, sleeve 64, and rod 65 therein can, can be inserted into the inferior lumen 62 of the cannula 71. Referring to FIG. 10B, the everting balloon catheter 71 can be advanced forward inside inferior lumen 62 of cannula 61 until its soft tip 72 lies distal to the distal tip 69 of cannula 61. For example, the rod 65 can be advanced into the catheter 71 to advance the balloon 63 and sleeve 64 toward and into the angled tips 69, 71. Referring to FIG. 10C the balloon 63 and sleeve 64 can be everted out of soft tip 72. For example, the rod 65 can be advanced into the angled tips 69, 71 to advance the balloon 63 and sleeve 64 outside of the angled tips 69, 71. In some embodiments, the rod 65 can be sufficiently radially flexible to contour with the angled tips 69, 71 while also being sufficiently linearly rigid to evert the balloon 63 and sleeve 64 outside of the angled tips 69, 71. The rod 65 can be radially flexible but sufficiently rigid to advance the balloon 63.

Referring to FIGS. 11A and 11B, in some embodiments, a cannula 81 can be used with an everting balloon catheter 91 that does not have a soft tip that extends distal to the end of the catheter 81, as discussed with respect to FIGS. 9-10C. The cannula 81 can be similar in design as discussed with respect to the cannulas 21, 41, 51. For example, the cannula 81 can include an inferior lumen 82, a superior lumen 86 housing an endoscope 87, an angled tip 89 and a transparent hood 88 similar to those same components discussed with respect to cannulas 21 41, 61. Similarly, the catheter 91 can include a balloon 83, a sleeve 84, and a rod 85 that operate in a similar manner as the balloons, sleeves, and rods discussed with respect to FIGS. 5A-10C. As shown in FIG. 11A, the inverted balloon 83 and inverted sleeve 84 can be everted from the distal end of catheter 91 a predetermined distance, for example, at a distance of approximately 10 mm-15 mm. Upon pressurization of catheter 91, the everted portion of balloon 83 and sleeve 84 can form a soft tip that possesses sufficient column strength to be inserted through the tubal os 18 into the Fallopian tube 19.

FIG. 11B shows the cannula 81 with an everted portion of balloon 83 and sleeve 84 advanced out of angled tip 89 of cannula 81, upon advancement of everting balloon catheter 91 within the inferior lumen 82 of cannula 81. For example, the rod 85 can be used to advance the balloon 83 and sleeve 84 through the angled tip 89 and out the end of the cannula 82. During clinical use, the angled tip 89 of cannula 81 can remains at the tubal os 18 as the everted portion of balloon 83 and sleeve 84 are manually advanced into the Fallopian tube 19. Following full length placement of the everted portion of balloon 83 and sleeve 84 past any potential outpouching at the tubal os 18, the balloon 83 can be fully everted to deploy sleeve 84 the length of the Fallopian tube 19.

In operation, the systems discussed with respect to FIGS. 5A-11B, can be used for cell collection within a biological vessel. The process can include placing, within the vessel, a cannula having an inverted balloon and an inverted sleeve situated within the inferior lumen of the cannula. The placing step can include guiding the cannula into position within the vessel (e.g., near os 18 or Fallopian tube 19) using a visual confirmation technique. For example, the hood can be at least partially inflated for endoscopic viewing from within the inferior lumen of the cannula during placement. When using endoscopic viewing, the hood can be inflated through the superior lumen or a separate lumen.

Once in place, the angle tip can be positioned near a target location (e.g., os 18 or pocket 20) in preparation of eversion of the sleeve, for example, as shown in FIG. 8B. The angled tip can be placed by manipulating the cannula (e.g., rotating, moving longitudinally, moving vertically, moving horizontally, etc.) In some instances, when using an everting catheter, the catheter can be at least partially everted from within the cannula. Thereafter, the inferior lumen can be pressurized, for example, by injecting a fluid therein. The pressurization of the inferior lumen can cause the balloon to be similarly pressurized and begin eversion of the balloon from the cannula and/or the catheter. Optionally, during the eversion of the balloon, the rate of eversion can be controlled through use of a rod or elongated member attached to a proximal end of the balloon.

Upon eversion of the balloon from the cannula and/or the catheter, the sleeve can also be everted from the cannula and/or the catheter, due to its relationship with the balloon. The balloon and the sleeve can be everted and can follow the contour of the vessel, for example, as shown in FIG. 8C. As the balloon and sleeve are everted, the sleeve can be positioned to initiate contact between a surface of the inverted sleeve and a sidewall of the vessel to collect a cell sample onto the surface of the sleeve. Thereafter, the balloon, sleeve, and optional catheter can be re-inverted within the cannula for removal. This can be performed by a combination of applying a negative to the inferior lumen, retracting the catheter, and/or retracting the rod. With the elements re-inverted into the cannula, the cannula can be removed. In some instances, the hood can remain at least partial inflated to provide assistance in the visual inspection during removal. At some point, the hood can be deflated for removal. Lastly, once removed, any collected cells (e.g., on the sleeve) can be obtained for analysis, testing, diagnostics, etc. using any combination of methods known in the art.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present disclosure be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A system for cell collection within a vessel, the system comprising:
a cannula having with a pathway extending between a first end and a second end of the cannula, and a first lumen situated longitudinally within the pathway of the cannula;
an inverted balloon situated within the first lumen, the balloon being coupled at one end to the second end of the cannula and coupled at an opposing end to a rod to permit controlled eversion in the presence of positive pressure along a defined path diverging from an axis of the pathway; and
an inverted sleeve situated within the balloon and being attached at one end to the second end of the cannula, such that it everts with the balloon along the defined path upon eversion of the balloon from the cannula.

2. The system of claim 1, wherein a surface of the inverted sleeve includes at least one of a textured surface, an adhesive surface, and an open mesh surface.

3. The system of claim 1, wherein the balloon is in fluid communication with the pathway and configured to receive pressurizing fluid from the pathway for eversion out from the second end of the cannula.

4. The system of claim 1, wherein the sleeve is configured to evert from the second end of the cannula upon pressurization of the balloon.

5. The system of claim 1, further comprising a second lumen within the cannula, the second lumen configured to receive an endoscope.

6. The system of claim 1, further comprising a transparent hood located at the second end of the cannula, the transparent hood configured to provide viewing from within the second end of the cannula.

7. The system of claim 1, wherein the cannula is sufficiently flexible to be advanced through a vessel in a body.

8. The system of claim 7, wherein the balloon has an expandable diameter sufficiently large to advance and press the inverted sleeve against inner walls of the vessel for cell collection.

9. The system of claim 8, wherein the vessel is a Fallopian tube.

10. The system of claim 1, wherein the inverted balloon and the inverted sleeve are configured to move longitudinally within the pathway of the cannula.

11. The system of claim 1, wherein the cannula is angled at its distal end in a direction radial away from the cannula to provide the defined path.

12. The system of claim 1, wherein the second end of the cannula includes an angled rounded tip.

13. A method for cell collection within a biological vessel, the method comprising:
placing within the vessel, a cannula having:
a pathway extending between a first end and a second end of the cannula and an inferior lumen positioned along the pathway;
an inverted balloon situated within the inferior lumen and being coupled to the second end of the cannula; and
an inverted sleeve situated within the balloon and being attached to the second end of the cannula, such that upon eversion of the balloon from the cannula, the sleeve is also everted from the cannula;
everting at least a portion of the inverted sleeve from the second end of the cannula; and
initiating contact between a surface of the inverted sleeve and a sidewall of the vessel to collect a cell sample onto the surface of the sleeve.

14. The method of claim 13, further comprising pressurizing the balloon by inputting fluid into the inferior lumen.

15. The method of claim 14, further comprising controlling eversion of the pressurized balloon by controlling an elongated member coupled to a proximal end of the balloon, to allow the balloon to evert longitudinally toward the second end of the cannula.

16. The method of claim 15, wherein the at least the portion of the sleeve is everted by the everting balloon.

17. The method of claim 16, wherein the sleeve is fully everted from the pathway of the cannula.

18. The method of claim 14, further comprising inflating a transparent hood located at the second end of the cannula.

19. The method of claim 18, further comprising advancing an endoscope in a superior lumen located within the cannula to view the second end of the cannula.

20. The method of claim 19, further comprising viewing the vessel via the endoscope through the transparent hood.
